(19) United States
(12) Patent Application Publication
(10) Pub. No.: US 2018/0052101 A1
(43) Pub. Date: Feb. 22, 2018

(54) Apparatus and Method for Measuring Components in Fluidic Samples Sealed in a Bag

(71) Applicant: Yuguang Cai, Lexington, KY (US)

(72) Inventor: Yuguang Cai, Lexington, KY (US)

(21) Appl. No.: 15/242,733

(22) Filed: Aug. 22, 2016

(51) Int. Cl. G01N 21/3577; G01N 21/03; G01N 21/11; G01N 33/14

(52) U.S. Cl. CPC G01N 21/3577; G01N 33/14; G01N 21/11; G01N 21/0303

(57) ABSTRACT

The present invention discloses an apparatus and a method for measuring components in fluidic samples in a non-invasive fashion using Infrared (IR) transmission spectroscopy. Fluidic samples are sealed in flexible IR-transparent bags that are then fixed on a supporting bed. The supporting bed is then mounted between the front and back plates of the apparatus so that the bag is squeezed by two IR transparent windows from opposite directions until the windows contact the spacer sheet mounted on the back plate. The thickness of the spacer sets the gap distance between the two windows and thereby sets the optical path for the measurement in the transmissive mode.

### REFERENCES CITED

### U.S. Patent Documents

| | | | |
|---|---|---|---|
| 5,510,621 | April 1996 | Goldman | 250/343 |
| 7,582,869 | September 2009 | Sting et al. | 250/336.1 |
| 6,280,690 | August 2001 | Tadion | 422/560 |
| 4,872,868 | October 1989 | Chevallier | 604/327 |
| 5,239,860 | August 1993 | Harris et al. | 73/61.48 |
| 7,952,710 | May 2011 | Flank et al. | 356/326 |

### OTHER PUBLICATIONS

EPA Method 418.1 Petroleum Hydrocarbons (Spectrophotometric, Infrared)
Rolene Bauer, Hilhne Nieuwoudt, Florian F. Bauer, Jens Kossmann, Klaus R. Koch, and Kim H. Esbensen, “FTIR Spectroscopy for Grape and Wine Analysis”, Analytical Chemistry 2008 80 (5), 1371-1379.
Shaw, R. A. and Mantsch, H. H. 2006. Infrared Spectroscopy in Clinical and Diagnostic Analysis. Encyclopedia of Analytical Chemistry.
Duarte, I. F. et al Journal of Agriculture and Food Chemistry, 2002, 50, 3104-3111.

### 1. BACKGROUND OF THE INVENTION

The present invention is an apparatus and a method for non-invasive, fast and economic analysis of fluid samples using Infrared (IR) transmission spectroscopy.

IR transmission spectroscopy has been widely employed to qualitatively identify and quantitatively assay the constituents in fluid media. For instance, EPA Method 418.1 specifies that the petroleum hydrocarbon contaminants in soil and water shall be extracted by fluorocarbons and then the extract fluid shall be analyzed by the IR absorption in transmission mode. In food industries, IR transmission spectroscopy has been routinely used for quality control. For instance, the article titled “FTIR Spectroscopy for Grape and Wine Analysis” by Bauer et al published on Analytical Chemistry, 1371, 2008 summarizes the applications of infrared transmission spectroscopy in wine analysis. In clinical analysis, Infrared transmission has been accepted as a powerful approach to analyze body fluids and medicinal fluids. (Shaw, R. A. and Mantsch, H. H. 2006. Infrared Spectroscopy in Clinical and Diagnostic Analysis. Encyclopedia of Analytical Chemistry.) U.S. Pat. No. 5,510,621 demonstrates a health-care industry application of IR transmission spectroscopy for analysis the total parenteral nutrients (TPN), which are eventually the source of intravenous feeding. TPN solution is a clear liquid, which has the components of saline water, amino acid and dextrose. The concentrations of TPN constituents are vital for patients' health. In hospitals, samples of TPN solution can be analyzed using the IR transmission spectroscopy method to verify the quality of the TPN solution to be fed to the patients.

In a standard procedure of IR transmission analysis, a certain amount of liquid is injected into an IR-transparent liquid cell, which is placed between an IR source and an IR detector. The IR beam passes the IR transparent cell windows perpendicularly. By measuring the absorption of light at the IR band, an IR spectrum is obtained, with peaks at frequencies corresponding to the specific chemical bond vibrations. The concentration of the sample can be computed by using the IR absorbance of the sample at a given wavelength. To achieve valid measurements, the liquid cell must contain a pair of optical grade flat windows, which is made of IR transparent materials such as ZnSe, CaF₂, NaCl, or KBr. The optical path is the distance between the two windows. Commercially available liquid cells for IR transmission spectroscopy are sold as demountable cells with adjustable optical path by changing spacers or as fixed optical path such as the SL2 sandwich cell from the International Crystal Laboratory, or as variable optical path cells such as the TumblIR®/Dialpath® from Agilent Technology Inc.

Major inconvenience and technical difficulty in IR analysis involving liquid samples arises from the direct liquid-window contact.

Due to the cost of the IR cell windows, disposable windows are not a practical solution, and the windows have to be reused. Therefore, in all current liquid cells on the market, the fluid samples directly contact the IR windows. As the result, the windows have to be cleaned thoroughly after each test to avoid cross-contamination for the subsequent testing using the same liquid cell. This recovering process is usually cumbersome and time-consuming.

Also, due to the sample-window direct contact, the selection of the window material is significantly restricted to those that do not react with or dissolve in the contacting liquid. For example, KBr windows, an economic broad-band IR-transparent window, are NOT compatible with aqueous solutions, because these solutions dissolve the KBr window. As the result, high-cost ZnSe or CaF₂ windows must be used, which has inferior performances (i.e. narrower-band) than KBr.

### 2. SUMMARY OF PRIOR ARTS

Established approaches for obtaining transmission spectra of liquids require direct liquid-sample holding cell contact. U.S. Pat. No. 7,582,869 describes a design a cell for obtaining transmission spectroscopy for liquids. The cell has two movable windows, between which is the chamber for holding liquids. Liquids can be introduced into the cell, and the optical path can be adjusted before measurement.

U.S. Pat. No. 6,280,690 describes methods and apparatus for obtaining transmission spectra of liquid and solid samples. In this disclosed method, liquid contacts a wire mesh at first. The liquid-soaked wire mesh is inserted into the sample holder of a spectrometer so that a beam of radiation passes the liquid remaining on the wire mesh and generates a transmission spectroscopy.

An approach that avoids direct liquid-cell contact is to use a flexible enclosure such as a bag or a tube to hold the sampling liquid and analyze the signal after the incident electromagnetic beam passes the enclosure. In this approach, a means is employed to ensure the pre-determined optical path is fixed during a test. The enclosure material must have a low absorption in the particular band of the electromagnetic wave of which the analytical method employs. With this approach, the liquid in the enclosure can be directly loaded/unloaded with the enclosure together without the need of rinsing since the liquid samples do not directly contact the optical components made of KBr, ZnSe or NaCl. Therefore, the analysis time for each sample can be reduced, and various liquid samples can be analyzed.

U.S. Pat. No. 4,872,868 shows an analyzer for collection bags which provides an envelope that permits the insertion of reagent's test strips and the like.

U.S. Pat. No. 5,239,860 describes a sensor for continuously measuring alcohol and gasoline fuel mixtures in a clear Teflon tube using a pre-determined optical path and electromagnetic radiation at a pair of wavelengths which are generated by rapidly switching currents through a light-source. Thermopile detectors are used to detect an increase in temperature due to light transmitted through the flowing gasoline/alcohol mixture.

U.S. Pat. No. 7,952,710 describes an apparatus and a method for detecting and quantifying constituents in solutions that are held in a bag by spectrometric methods.

U.S. Pat. No. 5,510,621 describes an apparatus for measuring components in liquid media, in particular, parenteral nutrients, within a flexible transparent bag. A threaded rod is utilized to adjust the optical path across the bag chamber and includes a passage for electromagnetic radiation of selected wavelengths. The source of electromagnetic radiation is capable of sending radiation into the bag chamber and to detector means which analyzes the radiation passed through or reflected from the components in the bag chamber.

This invention employs bags made of thin, low IR-absorbance film. The liquid sample is introduced into the bag and sealed. The bag is first fixed on a supporting bed then the bag-supporting bed assembly is placed into an apparatus in which two parallel IR-transparent windows are pushed against the bag from opposite positions until the pre-determined optical path is reached. The apparatus is then inserted into an IR spectrometer to analyze the liquid sample held between the windows. After the analysis, the bag is removed from the apparatus, and a new bag containing the next liquid sample will be loaded into the apparatus for the next analysis.

The invented apparatus and method enable the rapid analysis of multiple liquid samples, and reduction of the cost for analyzing solutions using IR transmission spectroscopy. The structure layout, the mechanism for setting the optical path the design of the bag, as well as the method of conducting sample preparation, loading, unloading are different from all prior arts.

This invention has fundamental differences with U.S. Pat. No. 7,952,710 in the following aspects:
1. The mechanism of setting optical path is different. This invention employs a pair of spacer sheet. In contrast, U.S. Pat. No. 7,952,710 uses a spring-loaded caliper to set optical path;
2. U.S. Pat. No. 7,952,710 is specifically designed for instruments that employ fiber optics as means for introducing incident beam of electromagnetic wave, which is indicated by the design of two ports in the apparatus for incident and outgoing optic fibers. The apparatus disclosed in this invention can be used with spectrometers with fiber optics or regular spectrometers that do not use optic fibers;
3. This invention employs a supporting bed to fix the flexible bag so that the deformation, slippage and wrinkling of the flexible bag can be prevented. U.S. Pat. No. 7,952,710 does not disclose such mechanism.

This invention has several differences with U.S. Pat. No. 5,510,621.

1. This invention differs from U.S. Pat. No. 5,510,621 in the designs and mechanism in setting the optical path in the following aspects:
1a) The field of application of U.S. Pat. No. 5,510,621 is the Near Infrared (NIR) and the apparatus is optimized for the typical NIR use. Specifically, the optical path for NIR transmission is 1-15 mm, as the U.S. Pat. No. 5,510,621 mentioned. This invention is optimized for the application in Mid-IR, which has a typical optical path of 0.01-1 mm. U.S. Pat. No. 5,510,621 sets the optical path by turning a threaded rod. In this invention, the optical path is set up using a series of spacer sheet, which have standard thickness from 0.01 to 1 mm.
1b) U.S. Pat. No. 5,510,621 changes the optical path from 0 to 25 mm by turning a threaded rod. There is no mechanism that prevents the rod from overtighten when the optical path is close to 0 mm, which may exert excessive force on the window and damage them. This invention uses the sheet spacer mounted in the elastic window holder to set optical path, which avoids this potential hazard.
1c) In the design of U.S. Pat. No. 5,510,621, by turning the threaded rod, the optical path can be increased or decreased. U.S. Pat. No. 5,510,621 offers no design to set the exact the optical path. To obtain the exact optical path, a caliper or experimental calibration work has to be employed to obtain the distance between the windows. In contrast, in this invention, the spacer sheet with known thickness is used to set the optical path.

This invention differs from U.S. Pat. No. 5,510,621 in the design of the bag.

In U.S. Pat. No. 5,510,621, the bag is directly placed inside the liquid cell, and the bag is open to the air. To load the bag into the apparatus, the bag has to rely on external suspension devices until the inner surfaces of 42 and 44 of fences 22 and 24 are pushed against the bag and provide sufficient friction to prevent bag slippage.

In this invention, two distinct mechanisms are employed to prevent the bag slippage and deformation.
2a) The bag is placed on a supporting bed and physically fixed on said bed. The weights of the liquid and the bag are supported by said spacer bed. Hence, during analysis, the bag stays vertically, and its surface remains fully stretched. No bag slippage occurs.
2b) The bag is sealed, the liquid sealed in the bag has a positive internal pressure, which renders the both bag walls to maintain a positive curvature. Said positive curvature ensures that when the two windows are pushed against the bag from opposite directions, bag wall that contacts the flat windows remains flat.
These designs avoid bag slippage and wrinkle formation on the bag surface. Said bag seals the liquid sample from the windows, the cell parts and the operators.

### 3. OBJECTIVES AND ADVANTAGES

The objectives of this invention are

1) to achieve rapid loading and unloading of liquid samples into IR spectrometer for fast analysis by loading and unloading bags sealed with sample liquid as a whole;
2) to reduce the analysis costs by using disposable sealed bags for holding liquid sample;
3) to remove the restriction on window material selection so that the low-cost and the boarder-band window material can be used for previously non-compatible liquid samples;
4) to protect the windows from damages associated with sample contacting;
5) to ensure measurements on reactive, unstable, corrosive, hazardous, contagious and filthy samples which were not suitable for the current IR transmission apparatus;
6) to set the optical path to a pre-determined value in the range of sub-millimeters (e.g. 0.01-1 mm).

This invention has the following advantages compared to prior arts.

Commercial available IR liquid cells such as the SL2 sandwich cell from the International Crystal Laboratory, or Omni-Cell from Specac Inc., Cranston, R.I., use the spacer to set the optical path. These cells are designed for directly inject liquid into said cell, where said liquid contacts the window and the ports. Therefore rinsing is required after each test. Flexible bags that holding sample liquid cannot be used in said liquid cell.
(a) This invention employs a design that uses the spacer sheet as a means to set the optical path.
(b) This invention employs a design that uses a flexible bag to hold the sample liquid. During IR transmission measurement, said bag is squeezed by two movable windows to reach said optical path set by the spacer.

U.S. Pat. No. 5,510,621 discloses an apparatus that use a bag to hold sample liquid, and the bag is placed between two windows, the distance between the windows can be adjusted by turning a threaded rod.

This invention combines said two designs, which are not simultaneously disclosed in U.S. Pat. No. 5,510,621 or demonstrated by any commercial available liquid cell on the market. The combination enables the rapid loading and unloading liquid samples by eliminating the time-consuming and high cost rinsing process, and to use the sealed bag with sample in a disposable way using Mid-IR analysis. The combination also enables the use of all IR window materials for analysis of various liquid samples, which reduces the total hardware cost for IR analysis. The combination also enables the use of reactive, unstable, corrosive, hazardous, contagious and filthy samples by isolating the samples from the other parts of the transmission cell and the operators. The low-cost and rapid analysis of multiple liquid samples using Mid-IR transmission spectroscopy, which is a long existed technical difficulty, can be overcome with this invention. Furthermore, liquid samples are sealed in bags before analysis. The sample loading process and analysis process can be separated and completed by different personnel. For analyst who operates the instrument, the training for liquid sample handling and personal protection equipment for that liquid may not be required. Another advantage is that the test is non-invasive and thus the sample can be recovered easily for further test, which is critical when the amount of sample is limited.

An apparatus and method for identifying solutions in a translucent transparent or semi-transparent bag, such as sugar in beverages, non-invasively, qualitatively and quantitatively would be a notable advance in the chemical analysis field.

### 4. SUMMARY OF THE INVENTION

This invention describes an apparatus and a method for non-invasive, fast analysis of fluid samples using IR transmission spectroscopy. The approach is to analyze liquid samples in flexible sealed bags. Said apparatus loads said bag in a fixed position and sets the pre-determined optical path for subsequent analysis. Said apparatus is composed of three parts, the front plate, the loading bed, and the back plate. The three parts are stack against each other in the order of the front plate, the loading bed, and the back plate and by means of fasteners such as bolts/nuts, springs, or magnets. Two windows are mounted on the center of the front and back plate. Said windows are made of material transparent to said electromagnetic radiation said instrument is employed. The sealed bag is physically attached to the loading bed. Next, said loading bed with said bag is mounted on the back plate. Holes on said loading bed and corresponding posts on said back plate are designed to align said loading bed to be placed exactly in the pre-determined position on said back plate. After the front plate is fastened to loading bed and the back plate, said apparatus is mounted between the source and the detector of an instrument that uses the transmittance of electromagnetic radiation as means of analysis. The sample is ready for analysis. After the measurement, said apparatus can be disassembled and the loading bed can be replaced by a new loading bed with the next sample mounted on it.

### 5. BRIEF DESCRIPTION OF THE DRAWINGS

FIG. 1 is the exploded view of the apparatus.

FIG. 2a. The exploded view of the back plate, from right side

FIG. 2b. The exploded view of the back plate, from left side

FIG. 3 The exploded view of the supporting bed

FIG. 4. The exploded view of the front plate, from left side

FIG. 5 The layout of the sample bag

FIG. 6 The scheme for loading and sealing liquid sample into the bag
a) Inject fluid sample into the bag via the port section; b) After fluid is accumulated in the cell section, squeeze the bag; c) The bag is squeezed until the liquid reaches the neck section; d) and e) The bag is then sealed.

FIG. 7 The scheme illustrating the process of installing a sample bag onto the apparatus
a. Select the correct pair of spacer sheet to set the optical path, and then mount the supporting bed to the back plate through the guiding posts.
b. Mount the bag onto the supporting bed through the guiding posts
c. Install the front plate
d. The apparatus with the sample installed, ready to be measured.

FIG. 8 The process of inserting the apparatus into a generic sample holder inside a spectrometer
a. Slide the apparatus down
b. The apparatus in position for measurement inside the spectrometer

FIG. 9 IR peak area of a series of sucrose solutions and their linear relationship with concentration
(a) IR transmission spectra of sucrose solutions with different concentrations (spectral range 1300-1000 cm⁻¹), the samples were in polyethylene bags with a constant OP of 25 μm. (b) Sucrose concentration vs. Absorbance for the band area at 1050 cm⁻¹.

FIG. 10 IR spectra of toluene with different optical path and the relationship between the peak area and corresponding optical path

(a) IR transmission spectra of Toluene (spectral range 2200-1650 cm⁻¹)), the samples were in polyethylene bags and tested with different OP (i.e. various thick spacers) (b) Absorbance vs. Optical path curve for the band area at 1952 cm⁻¹. A least-square fitting line is plotted, and the coefficient of determination (R²=0.996) is displayed as well.

For a better understanding of the invention, reference is made to the following detailed description of the preferred embodiments which should be referenced to the herein before described drawings.

### 6. DETAILED DESCRIPTION OF THE PREFERRED EMBODIMENTS

Various aspects of the present invention will evolve from the following detailed description of the preferred embodiments thereof which should be taken in conjunction with the hereinbefore described drawings.

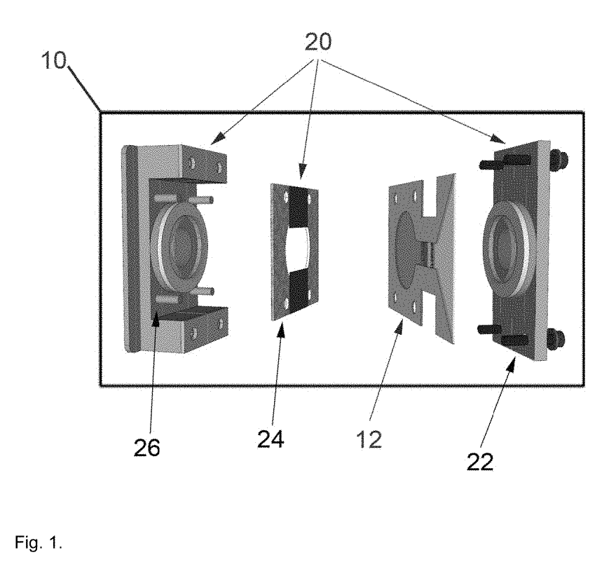

The invention as a whole is depicted in the drawings by reference character 10. The invention is composed of an apparatus 20 and a bag 12, which can be mounted and dismounted from 20. The apparatus 20 is shown in the drawings as including three embodiments, a front plate 22, a supporting bed 24, and a back plate 26. Referring to FIG. 1, during measurement, the flexible bag 12 is mounted on the supporting bed 24. The back plate 26, the supporting bed 24 with the mounted bag 12 and the front plate 22 are held against each other by means of fasteners, spring-loaded hinges, spring-loaded clips, clamps, magnets or friction.

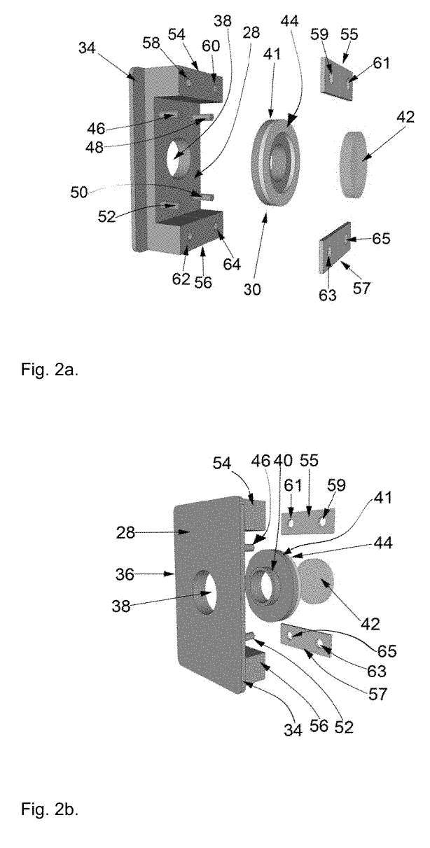

FIG. 2a shows the layout of the back plate 26, which is viewed from right side. The back plate 26 is composed of a back chassis 28, a window holder 30, and a window 42. The drawing in FIG. 2b shows that the back chassis has a rectangular shape from the left viewpoint. The back chassis has two wings 34, 36 in the left and right sides of the back chassis. Wings 34, 36 are used to guide the apparatus 20 to slide into the alignment slots of standard sample holders of a spectrometer. A round hole 38 in the center of the back chassis 28 is designed for mounting windows on the back chassis 28. The window holder 30 is made of elastomer. The shape of the window holder 30 can be depicted as three fused co-axial tubes 40, 41, 44. The outside diameter of the smallest tube 40 is the same as the diameter of hole 38. Tube 40 is inserted into hole 38. Thereby the window holder 30 is mounted on the back chassis 28. Tubes 40 and 41 have the same inside diameter. The outside diameter of tube 41 and 44 are the same, which is larger than the diameter of the window 42. The inside diameter of tube 44 is same as the diameter of the window 42, but it is larger than the inside diameter of the smaller tube 40. Window 42 is held by tube 44 when it is mounted on window holder 30. Window 42 is made of materials that have a low Mid-IR absorbance such as KBr, NaCl, ZnSe, Si, CaF₂ or Ge. On the back chassis 28, four posts 46, 48, 50, 52 are located around hole 30. Posts 46, 48, 50, 52 are symmetric with respect to the center of hole 30 and they are arranged in a rectangle shape. The border lines of this rectangle formed by posts 46, 48, 50, 52 are parallel to the borderlines of the back chassis 28. Posts 46, 48, 50, 52 are perpendicular to the flat surface of window 42. Posts 46, 48, 50, 52 function as the alignment guiding rods for hooking up supporting bed 24, which has four holes in the corresponding positions. Two spacer bars 54, 56 are located along the borders of the back chassis 28. Spacer bars 54 and 56 are perpendicular to the wings 34, 36. Spacer bar washers 55, 57 are on top of spacer bar 54, 56, respectively. Spacer bar washers 55, 57 are made of elastomer. There are four tapped holes 58, 60, 62, 64 on the spacer bars 54, 56. Each bar has two holes. There are four through holes 59, 61, 63, 65 on the spacer bar washer 55, 57. Each bar washer has two holes. Bolts are inserted into holes 58, 60, 62, 64 to assemble back plate 26, supporting bed 24 and front plate 22.

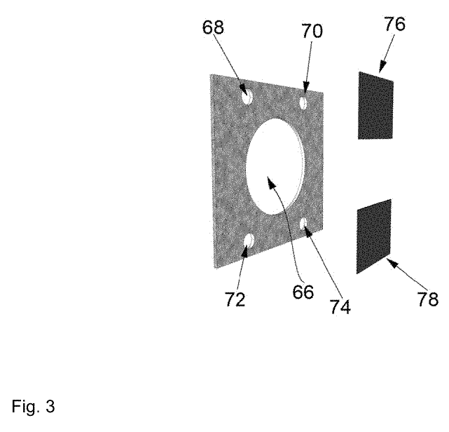

FIG. 3 shows the layout of supporting bed 24. Supporting bed 24 is a thin sheet with a large round cavity 66 in the center. The diameter of cavity 66 is larger than the diameter of window 42, 44. The center of cavity 66 and center of window 42 are on the same axis as tubes 40, 41, 44. Four holes 68, 70, 72, 74 are located on supporting bed 24. The distances from the centers of holes 68, 70, 72, 74 to the center of cavity 66 are the same. Lines connecting the centers of holes 68, 70, 72, 74 form a rectangle. The diameter of holes 68, 70, 72, 74 is the same as the diameter of posts 46, 48, 50, 52. The distance between the centers of holes 68, 70, 72, 74 are the same as the distance between the centers of posts 46, 48, 50, 52. Two sheet spacers 76, 78 are attached along the border lines of supporting bed 24. Sheet spacers 76, 78 adopt the same shape as spacer bars 54, 56 and are aligned in the same directions as spacer bars 54, 56. The width of sheet spacers 76, 78 determines the gap between sheet spacer 76, 78. This gap is designed to be smaller than the diameter of window 42. Sheet spacers 76, 78 are designed to set the optical path, which have exactly the same thickness and their thickness determines the distance between windows 42, 84 when they are pushed against each other from opposite directions. Sheet spacers 76, 78 are made of ultra-flat sheet with a series of standard thickness such as 10 μm, 20 μm, 30 μm, 50 μm, 100 μm, 200 μm and so on. By choosing a pair of sheet spacers 76, 78 with designated thickness, the optical path is set accordingly.

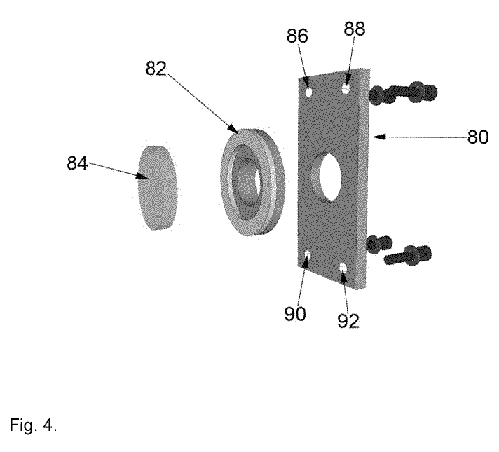

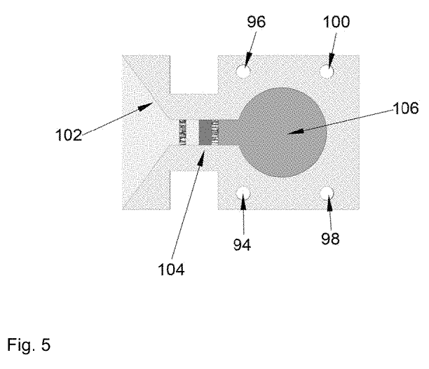

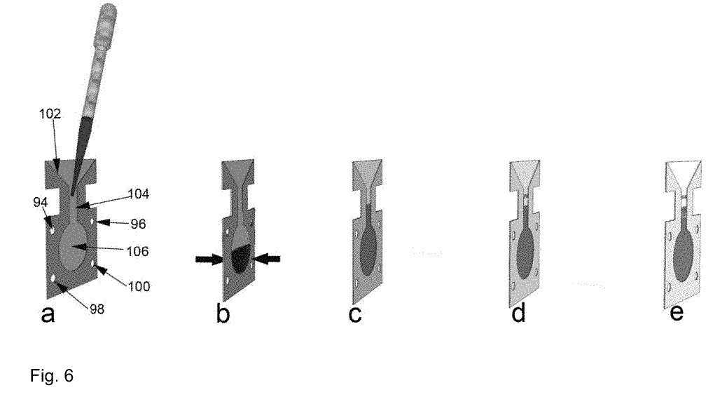

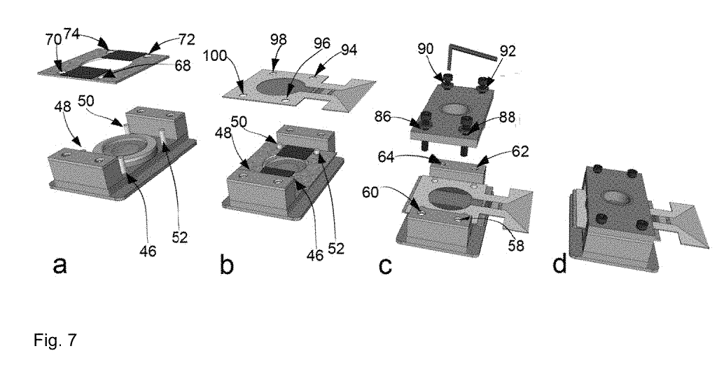

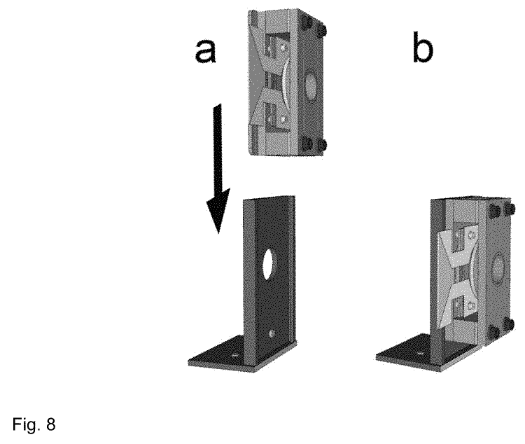

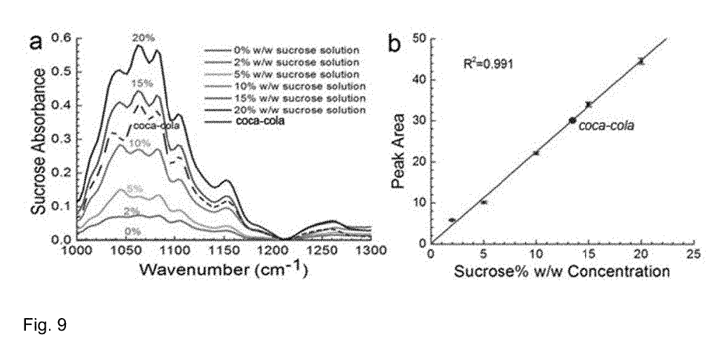

The front plate 22 is composed of a front chassis 80, a window holder 82, and a window 84. The drawing of 80 in FIG. 4 shows that the front chassis has a rectangular shape. Window holder 82 is the same as window holder 30. Window 84 is identical to window 42. Front chassis 80, window holder 82, and window 84 are mounted in the same way as the assembly of back chassis 28, window holder 30, and window 42. Four through holes 86, 88, 90, 92 are located at the four corners of front chassis 80. The diameter of holes 86, 88, 90, 92 is the same as the diameter of holes 58, 60, 62, 64. Four bolts are inserted into holes 86, 88, 90, 92, and are fastened in holes 58, 60, 62, 64, respectively.

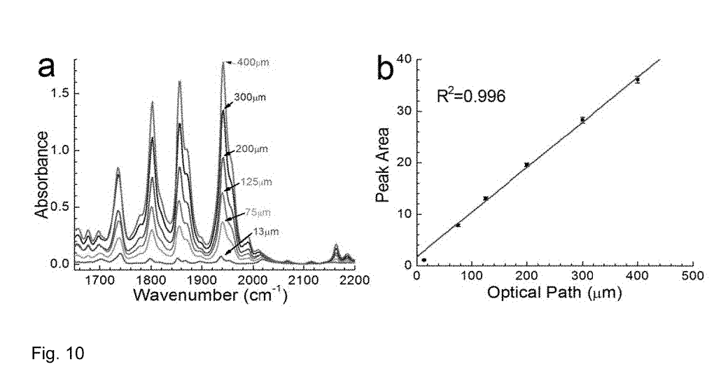

The shape of bag 12 is illustrated in FIG. 5. The bag is formed by fusing two pieces of thin film together. The means of fusing includes thermos-fusing, press-fusing, adhesive glue, or stitching by wires. Four holes 94, 96, 98, 100 are located on the four corners of the bag. The diameter of holes 94, 96, 98, 100 is the same as the diameter of posts 46, 48, 50, 52. The liquid-holding section of bag 12 is composed of a port 102, a neck 104 and a cell 106.

### 7. DESCRIPTION OF OPERATION

Loading Liquid into the Bag

The loading process is illustrated in FIG. 6. Bag 12 is placed in the direction that port 102 is up, and cell 106 is down. A pre-determined amount of liquid sample is injected into bag 12 through the opening port 102. Next, cell 106 is squeezed gently from the two window areas so that the liquid meniscus inside bag 12 reaches to neck 104. Then a means of sealing, such as thin plastic film thermal sealer, clamps, clip, magnate, glue or stitch, is used to close the bag at port 102 section. The sealing can be conducted once or multiple times at different positions in port 102 section so that no air is remained in the sealed bag. The volume of bag 12 is zero before liquid is injected and the flexible thin film is fully extended with no tension. After liquid injection, because of the volume of liquid sealed in bag 12, bag 12 forms a positive curvature over cell 106.

### Mounting the Sealed Bag on Back Plate 26

Four posts 46, 48, 50, 52 on the back chassis 28, four holes 68, 70, 74, 72 on supporting bed 24, and holes 96, 100, 98, 94 on bag 12 are aligned according to FIG. 7. Next, supporting bed 24 is pushed to let posts 46, 48, 50, 52 inserted into corresponding holes 68, 70, 74, 72 so that supporting bed 24 is fixed on back chassis 28. Next, bag 12 is pushed to let posts 46, 48, 50, 52 inserted into corresponding holes so that bag 12 is fixed to avoid bag slippage and bag collapse.

### Assembling the Apparatus 20

Front plate 22 is pushed against back plate 26. A typical means of pushing is to use four long bolts to fasten front plate 22 and back plate 26. Four threaded bolts are inserted into through holes 86, 88, 90, 92 on front plate 22, then into four through holes 61, 59, 65, 63 on spacer bar washers 55, 57 and ended in tapped holes 60, 58, 64, 62 on back plate 26.

Because front plate 22 is pushed against back plate 26, window 42 and window 84 are moved against each other until window 42 and window 84 contact sheet spacers 76, 78. Sheet spacers 76, 78 are sandwiched between windows 42 and 84. A further pushing of windows 42 and 84 causes the elastomer window holders 30, 82 to deform. The elastic deformation of windows holders 30, 82 maintains the distance between windows 42 and 84 and prevents the window from cracking due to excessive pushing forces.

Installing the Apparatus into Spectrometer

FIG. 8 shows that apparatus 20 is inserted into the standard sample holder in a generic spectrometer by aligning wings 34, 36 of apparatus 20 with the slots in the sample holder and then slide the apparatus into the sample holder. A beam of electromagnetic radiation then passes through the sample loaded inside bag 12 and reaches the detector.

It will be understood by those skilled in the art that while an embodiment of the invention was disclosed in considerable detail for purposes of illustration, many of these details may be varied without departing from the spirit and scope of the invention.

### 8. EXAMPLES OF APPLICATION

### Example 1

### Determine the Sucrose Concentration of Regular Coca-Cola.

Six standard solutions containing 0%, 2%, 5%, 10%, 15% and 20% w/w sucrose in distilled water are prepared. These solutions are sealed in the bag and loaded on the apparatus, respectively. The optical path is set to 25 μm and the Mid-IR transmission spectra are acquired for each sample, which are shown in FIG. 9a. The band near 1050 cm⁻¹ reflects the O-C stretching in sucrose molecules, and its peak area is used to assay the concentration of sucrose. [ref. Duarte, I. F. et al Journal of Agriculture and Food Chemistry, 2002, 50, 3104-3111.] The peak area versus concentration is plotted in FIG. 9b. The fitting shows that the concentration and the corresponding peak area demonstrate a linear relationship, which follows the Lambert-Beer's law. The least square fitting of the data points in FIG. 9b yields that the coefficient of determination (R²) is 0.991, the slope is 2.23±0.02 and the intercept is 0.12±0.06. Next, a sample coca-cola with unknown sucrose content is also analyzed in the IR spectrometer using the same setup. Its peak area at 1050 cm⁻¹ is 29.5. Using the standard calibration curve plotted in FIG. 9b, we determine that the sucrose concentration in the sample coca-cola is 13.17±0.12% w/w.

### Example 2

Demonstrate that the Absorbance of Toluene is Linearly Dependent on the Set Optical Path of the Apparatus

The Lambert-Beer's law states that the absorbance is a linear function of the optical path. In this experiment,
1. We set the optical path of the apparatus to 13 μm, 75 μm, 125 μm, 200 μm, 300 μm, and 400 μm;
2. We loaded toluene in sealed bag into the apparatus;
3. We measured the corresponding absorbance of toluene sealed in a bag.

The obtained toluene mid-IR spectra in the 1650 cm⁻¹-2200 cm⁻¹ range are plotted in FIG. 10a. The area of the peak at 1952 cm⁻¹ is used to represent the absorbance of toluene. We then plotted the peak area as a function of the set optical paths in FIG. 10b. The least square fitting of data points in FIG. 10b yields the coefficient of determination (R²) to be 0.996, which indicates the good linearity. Such excellent linearity of the plot demonstrates the accuracy and precision of the optical path setting mechanism of this invention.

I claim:

1. An apparatus for analyzing fluid samples using the transmission spectroscopy of electromagnetic radiations, comprising:
(a) A supporting bed for holding a sample bag to a fixed position on said supporting bed so as to prevent said bag from slipping or deforming;
(b) A back plate for holding said supporting bed at the fixed position;
(c) A front plate for holding said supporting bed at the fixed position;

2. Said apparatus of claim 1 in which said supporting bed is sandwiched between said front plate and said back plate. Said front plate and said back plate are fastened so that said supporting bed is squeezed by said front plate and said back plate from two opposite directions.

3. Said apparatus of claim 2 employs means for fastening said front plate and said back plate. Said means of fastening includes bolts, spring-loaded wires, spring-loaded clip, glue, friction force, and magnetic force.

4. Said apparatus of claim 1 in which said supporting bed has a void in the center so that the beam of electromagnetic radiations can pass. Said supporting bed also has four holes in four corners for aligning said supporting bed with said back plate.

5. Said apparatus of claim 1 in which said back plate comprises a back chassis, a window holder, and a piece of window.

6. Said window of claim 5 is made of materials with low absorption of said electromagnetic radiations.

7. Said window holder of claim 5 is made of elastomer. Said window of claim 5 is mounted in said window holder.

8. Said back chassis of claim 5 has a void in the center so that said windows holder in claim 5 is mounted in said void.

9. Said back chassis of claim 5 has four posts perpendicular to said back chassis surface. Said posts align to said holes of claim 4 when said supporting bed in claim 1 is pushed toward said back plate of claim 1 so that said supporting bed is held in a fixed position.

10. Said apparatus of claim 1 in which said front plate comprises a front chassis, a window holder, and a piece of window.

11. Said window of claim 10 is made of materials with low absorption of said electromagnetic radiations.

12. Said window holder of claim 10 is made of elastomer. Said window of claim 10 is mounted in said window holder.

13. Said front chassis of claim 10 has a void in the center so that said windows holder in claim 10 is mounted in said void.

14. Said apparatus of claim 1 employs a pair of sheet spacers to set the optical path before obtaining the transmission spectroscopy of said electromagnetic radiations. Said sheet spacers are flat sheet with pre-determined thickness. Said sheet spacer in each pair has identical thickness. Said pair of spacers is placed on the upper and lower section of said void of claim 4, respectively. In operation, said back plate, said supporting bed, and said front plate are fastened against each other. Said pair of sheet spacers is sandwiched between said window of claim 5 and said window of claim 10. Therefore, the distance between said window of claim 5 and said window of claim 10 is the thickness of said sheet spacer, which sets the optical path. A series of sheet spacer pairs with different thickness is available to set the optical path to different values. Therefore, said apparatus of claim 1 sets optical path to a series of pre-determined values with no need for adjustment and calibration.

15. Said sheet spacers of claim 14 can be cut into a plural of pieces or joined together to form a one-piece sheet spacer.

16. A bag for holding fluidic sample in the transmission measurement of electromagnetic radiations.

17. Said bag of claim 16 is made of a flexible film that has a low absorption in one or a plural of bands of said electromagnetic radiations.

18. Said bag of claim 16 has four holes in its four corners. Said poles of claim 9 are inserted into said holes, so that said bag is fixed on said apparatus.

19. Said bag of claim 16 comprises one or a plural of port sections, one or a plural of neck sections, and one cell section. Said port is a wide mouth that receives the fluid sample. Said neck provides a narrow channel that leads fluid to cell section. Said cell holds fluid. Said electromagnetic radiation passes through said cell, interacts with the fluid holding inside said bag before it reaches the detector.

20. A method for rapid loading and unloading fluid samples in a spectrometer that operates in transmission mode of electromagnetic radiation, comprising steps of:
a. Fluid sample is injected into said bag of claim 17, via said port of said bag so that said fluid is accumulated in said cell section.
b. Bag walls of said cell are squeezed from opposite directions so that fluid level in said cell reaches to said neck section.
c. Said bag is closed at the jointing section between said cell and said neck by means of sealing, which includes thermos-fusing, glue, clip-holding, stitching with wires, tie with wires, bending, or combination of heretofore means. Next, said bag is closed again at the jointing section between said neck and said port.
d. Said sealed bag is placed on said support bed of claim 1.
e. Said pair of sheet spacers is positioned on said support bed to set optical path.
f. Said back plate of claim 1, said supporting bed of claim 1, said sealed bag, said front plate of claim 1 are fastened together.
g. Said apparatus of claim 1 is mounted in a spectrometer for measurement.
h. After measurement, said apparatus of claim 1 is un-fastened and said bag is removed.
